(19) United States
(12) Patent Application Publication
(10) Pub. No.: US 2026/0124057 A1
(43) Pub. Date: May 7, 2026

(54) MUSCULOSKELETAL APPARATUS, METHODS, AND SYSTEMS OPERABLE TO ADMINISTER ENERGY PRESCRIPTIONS AND VERIFY THEIR EFFICACY

(71) Applicant: DATAFEEL INC., WASHINGTON, DC (US)

(72) Inventor: Matthew Robert Leaper, Omaha, NE (US)

(73) Assignee: DATAFEEL INC., WASHINGTON, DC (US)

(21) Appl. No.: 19/119,035

(22) PCT Filed: Oct. 9, 2023
(86) PCT No.: PCT/US2023/034768

§ 371 Date: Apr. 7, 2025

Related U.S. Application Data
(60) Provisional application No. 63/414,140, filed on Oct. 7, 2022.
(60) Provisional application No. 63/464,537, filed on May 5, 2023.

(51) Int. Cl. A61F 5/01; A61F 7/00; A61H 23/02; A61K 41/00; A61M 5/32

(52) U.S. Cl. CPC A61F 5/0123; A61F 7/007; A61H 23/0218; A61K 41/0047; A61M 5/32; A61F 2007/0042; A61H 2205/102; A61M 2202/04; A61M 2210/086

(57) ABSTRACT

Aspects of musculoskeletal apparatus, methods, and systems are disclosed. One aspect includes a brace that is wearable on a limb and operable to guide and support movements of a muscoskeletal joint of the limb; a plurality of multi-energy generators that are attachable to the brace at different treatment sites about the joint, each multi-energy generator comprising: a plurality of generator elements operable to output a plurality of different energy types toward the joint at one of the treatment sites, each generator element being operable to output one of the plurality of different energy types, and a heat sink that dissipates excess heat into air surrounding the limb; and a structure that attaches the multi-energy generators to the brace and is operable when the brace is worn to press the energy generators toward the different treatment sites during the movements of the joint. Related apparatus, methods, and systems also are described.

### CROSS-REFERENCE TO RELATED APPLICATIONS

This application is a § 371 National Stage Entry of International Patent App. No. PCT/US23/34768, filed Oct. 9, 2023, claiming the benefit of priority of U.S. Provisional Patent App. No. 63/414,140, filed May 5, 2023, and U.S. Provisional Patent App. No. 63/464,537, filed Oct. 7, 2022, the entireties of which are incorporated by reference into this application.

### TECHNICAL FIELD

Aspects of the present disclosure generally relate to musculoskeletal apparatus, methods, and systems. Some aspects are described with reference to examples operable to administer energy prescriptions and verify their efficacy.

### BACKGROUND

Many musculoskeletal injuries cannot heal effectively without immobilization and restraint. For injuries associated with a joint like the knee, for example, it is common to deploy a structural brace that limits lateral movements of the superior leg relative to the inferior leg in the coronal plane while permitting and often guiding walking movements of the superior leg relative to the inferior leg in the sagittal plane. Various medications are often prescribed and used to treat pain associated with the injury, making it easier for the injured party walk with support of the structure brace. It is known that some of these medications can be habit forming, such as opiates, potentially causing more harm than good. It is also known that a thermal energy like cold from an ice pack may be applied to treat the pain. Further improvements are required to enable and promote the prescription of additional forms of energy-based treatments or “energy prescriptions” as viable alternatives to medication-based treatments.

### SUMMARY

Numerous aspects are described in this disclosure. One aspect of this disclosure is a system. The system may comprise a brace that is wearable on a limb and operable to guide and support movements of a muscoskeletal joint of the limb; a plurality of multi-energy generators that are attachable to the brace at different treatment sites about the muscoskeletal joint, each multi-energy generator of the plurality of multi-energy generators comprising: a plurality of generator elements operable to output a plurality of different energy types in a signal direction toward the muscoskeletal joint at one treatment site of the different treatment sites, each generator element of the plurality of generator elements being independently operable to output one energy type of the plurality of different energy types, and a heat sink that dissipates excess heat generated from the plurality of generator elements into air surrounding the limb; and a structure that attaches the plurality of multi-energy generators to the brace and is operable when the brace is worn to press the plurality of energy generators toward the different treatment sites during the movements of the joint.

The brace may comprise a first frame wearable on one side of the muscoskeletal joint, a second frame wearable on an opposite side of the muscoskeletal joint, and one or more hinges operable to guide and support the movements of the muscoskeletal joint by limiting rotational movements of the first frame relative to the second frame. The system may comprise a power source for the plurality of multi-energy generators that is mounted to and contain in a structural member of the frame. The brace may comprise an open network of beam elements operable to obtain a form-fit with the muscoskeletal joint. The brace may comprise one or more living hinges operable to guide and support the movements of the muscoskeletal joint by limiting rotational movements of the joint. The system may comprise a sleeve that is wearable under the brace and comprises openings located approximate to the different treatment sites. The openings may comprise: a plurality of interface openings sized to receive skin-contacting surfaces of the plurality of generators, and at least one injection opening that is located to permit injection of a fluid through the sleeve and into the muscoskeletal joint at a location under one or more of the plurality of openings.

The plurality of different energy types may comprise a thermal energy and the plurality of generator elements of each multi-energy generator of the plurality of multi-energy generators comprise a thermoelectric cooler that is operable to output the thermal energy and thermally coupled to the heat sink. Each multi-energy generator of the plurality of multi-energy generators may comprise a PCB board; and a skin-facing side of the thermoelectric cooler may be positioned toward the skin, an outward-facing side of the thermoelectric cooler may be positioned away from the skin and thermally coupled to the heat sink, and a portion of the thermoelectric cooler may be located between its skin-facing and outward-facing sides is attached to a perimeter edge of the PCB board. The thermoelectric cooler comprises an annular perimeter shape and an I-shaped cross section defined by first annular ring, a second annular ring, and an array of Peltier coolers located between the first annual ring and the second annual ring. The heat sink may comprise a plurality of fins extending outwardly therefrom.

The structure may comprise an elastic portion that is structurally attached to the brace and resiliently operable to press the plurality of energy generators toward the different treatment sites during the movements of the muscoskeletal joint when the brace is worn. The structure may comprise a metallic portion that is thermally coupled to the frame and each heat sink of the plurality of multi-energy generators. The structure may comprise a plurality of cords and a plurality of nodes, the plurality of multi-energy generators may be mounted in the plurality of nodes, and the plurality of cords may attach the plurality of nodes to the frame and be resiliently expandable and contractable the press the plurality of multi-energy generators toward the different treatment sites. The plurality of multi-energy generators may comprise skin-contacting surfaces that are pressed toward the different treatment sites by the plurality of cords. The plurality of different energy types may comprise a vibratory energy and, for each multi-energy generator of the plurality of multi-energy generators, the plurality of generator elements may comprise a linear resonate actuator or a piezoelectric actuator that is operable to output the vibratory energy through a skin-contacting surface of the multi-energy generator.

The system may comprise a treatment delivery device operable with an exterior surface of one node of the plurality of nodes to deliver a fluidic treatment to an injection site below the one node when the brace is worn. The treatment delivery device may comprise a body defining: a contact surface positionable against the exterior surface of the one node; and a needle guide operable to guide a tip of a needle to the injection site. The contact surface may be curved to match a curvature of the exterior surface of the one node. The system may comprise a fluidic treatment that is deliverable to the injection site with the needle and comprises a biologic-based treatment, a chemical-based treatment, and a pharmacological-based treatment. The fluidic treatment may be affected by at least one energy of the plurality of different energy types. The fluidic treatment may comprise: a biocompatible gel, and a plurality of microspheres containing separate portions of the fluidic treatment; and each microsphere of the plurality of microspheres may be operable to release its separate portion of the fluidic treatment responsive to at least one energy of the plurality of different energy types.

The plurality of microspheres may comprise: a first set of microspheres operable to release a first volume of the fluidic treatment responsive to an output of a first energy of the plurality of different energy types; and a second set of microspheres operable to release a second volume of the fluidic treatment responsive to either: a different output of the first energy, or an output of a second energy of the plurality of different energy types. The system may comprise a controller that is remotely operable to cause the plurality of microspheres to release their separate portions of the fluidic treatment by causing one or more generators of the plurality of generators to output the at least one energy toward the muscoskeletal joint. The system may comprise a sensor operable to output data associated with a usage of brace or an efficacy of the fluidic treatment.

By way of example, the limb may be a leg, the muscoskeletal joint may be a knee, and the plurality of different treatment sites may be located approximate to one or more of the patella, the ACL, the MCL, and the pes anserine.

Related apparatus, kits, methods, and systems also are described.

### DESCRIPTION OF THE DRAWINGS

The accompanying drawings are incorporated in and constitute a part of this specification. These drawings illustrate exemplary aspects of the present disclosure that, together with the written descriptions provided herein, serve to explain the principles of this disclosure. Numerous aspects are shown conceptually in the drawings and particularly described, pointed out, and taught in the written descriptions. Some structural and operational aspects may be better understood by referencing the written portions together with the accompanying drawings, of which:

FIG. 1 depicts an exemplary system comprising a brace, sleeve, a plurality of energy generators, and structure attaching the generators to the brace;

FIG. 2 depicts another view of the FIG. 1 system;

FIG. 3 depicts another view of the FIG. 1 system;

FIG. 4 depicts a close-up view of portion of the FIG. 1 structure containing one energy generator of the FIG. 1 system;

FIG. 5 depicts another view of the FIG. 4 structure and energy generator;

FIG. 6 depicts a cross-sectional view of the FIG. 4 structure and energy generator;

FIG. 7 depicts another cross-sectional view of the FIG. 4 structure and energy generator;

FIG. 8 depicts a cross-sectional view of the FIG. 4 energy generator;

FIG. 9 depicts top and cross sectional views of the FIG. 4 energy generator;

FIG. 10 depicts top and cross sectional views of another energy generator;

FIG. 11 depicts the FIG. 1 brace;

FIG. 12 depicts the FIG. 1 sleeve;

FIG. 13 depicts the FIG. 1 structure;

FIG. 14 depicts the FIG. 1 structure and plurality of energy generators;

FIG. 15 depicts additional views of the FIG. 1 system;

FIG. 16 depicts another exemplary system comprising a brace, sleeve, a plurality of energy generators, and structure attaching the generators to the brace;

FIG. 17 depicts a side view of the FIG. 17 system;

FIG. 18 depicts a close-up view of the FIG. 17 system;

FIG. 19 depicts another exemplary system comprising the FIG. 17 system and a treatment delivery apparatus operable therewith;

FIG. 20 depicts perspective, top, and side views of the FIG. 17 delivery apparatus after being positioned against one energy generator of the FIG. 16 system;

FIG. 21 depicts a side view of the FIG. 17 delivery apparatus after being positioned against the energy generator; and

FIG. 22 depicts a side view of the FIG. 17 delivery apparatus after being positioned against the energy generator together with a cross-sectional view of an exemplary muscoskeletal joint.

Aspects of the examples illustrated in the drawings may be explained further by way of citations to the drawing and element numbers in the text of the description. The drawings and any citations thereto are provided for illustration purposes, and to further clarify the description of the present disclosure and are not intended to limit the present disclosure unless claimed.

### DETAILED DESCRIPTION

Aspects of the present disclosure are not limited to the exemplary structural details and component arrangements described in this description and shown in the accompanying drawings. Many aspects of this disclosure may be applicable to other aspects and/or capable of being practiced or carried out in various variants of use, including the examples described herein. Any example or variation may be claimed.

Throughout the written descriptions, specific details are set forth in order to provide a more thorough understanding to persons of ordinary skill in the art. For convenience and ease of description, some well-known elements and methods are described conceptually to avoid unnecessarily obscuring the focus of this disclosure. In this regard, the written descriptions and accompanying drawings should be broadly interpreted as illustrative rather than restrictive, enabling rather than limiting.

Exemplary aspects of this disclosure reference musculoskeletal apparatus, methods, and systems operable to administer energy prescriptions and verify their efficacy. As described herein, exemplary terms such as “energy generator,” “lattice structure,” “wearable structure,” and the like may be defined with reference to one or more of U.S. Pat. No. 10,959,674 and its progeny, U.S. patent Ser. No. 17/797,361 and its progeny, U.S. patent Ser. No. 18/024,371 and its progeny, U.S. patent Ser. No. 17/922,791 at its progeny, U.S. Prov. Pat. App. No. 63/414,140 and its progeny, and U.S. Prov. Pat. App. No. 63/356,950 and its progeny, the entireties of which including their progeny are hereby incorporated into this disclosure. Unless claimed, these exemplary aspects are provided for convenience and not intended to be limiting.

Several different reference axes are described, including a longitudinal axis and a lateral axis. Relevant arrangements may be described in relation to the different reference axes. For example, the longitudinal axis may be non-parallel with the lateral axis in some perspectives, meaning that one axis extends across the other. Relative terms such as “long” and “elongated” may describe any aspect having a length along one reference axis (e.g., the longitudinal axis) that is longer in relation to a width along a non-parallel reference axis (e.g., the lateral axis). Anatomical terms such as “anterior” and “posterior,” “medial” and “lateral,” and “proximal” and “distal” may be used to describe some structures in relation to a reference axis. For example, the longitudinal axis may be parallel to a bone structure of a user (e.g., an inferior arm) and extend between a proximal end of the bone structure (e.g., an elbow) and a distal end of the bone structure (e.g., a hand), making it a proximal-distal axis. Movements and forces may be similarly described in relation to any reference axis. As before, the different reference axes and any terms associated therewith are provided for convenience and not intended to limit this disclosure unless claimed.

As used herein, inclusive terms such as “comprises,” “comprising,” “includes,” “including,” and variations thereof, are intended to cover a non-exclusive inclusion, such that any wearable data communication apparatus, kits, methods, system, or element thereof that is described herein as comprising an exemplary list of elements does not include only those elements but may include other elements not expressly listed and/or inherent thereto. Unless stated otherwise, the term “exemplary” is used in the sense of “example,” rather than “ideal,” and does not limit this disclosure to any particular embodiment. Various terms of approximation may be used in this disclosure, including “approximately” and “generally.” Unless stated otherwise, approximately means within 10% of a stated number or outcome and generally means “within most cases” or greater than 50% chance.

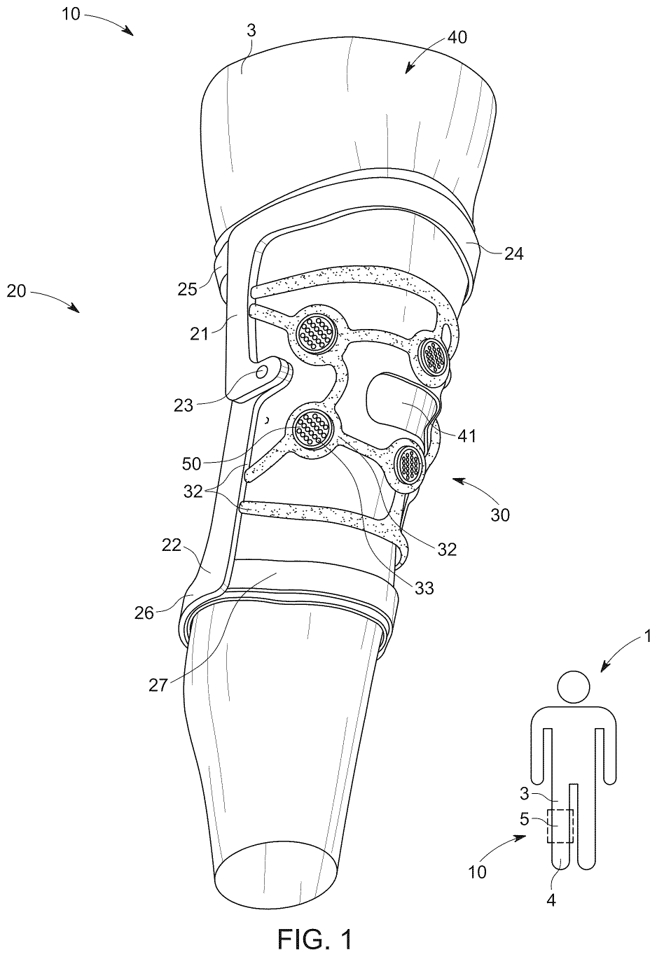

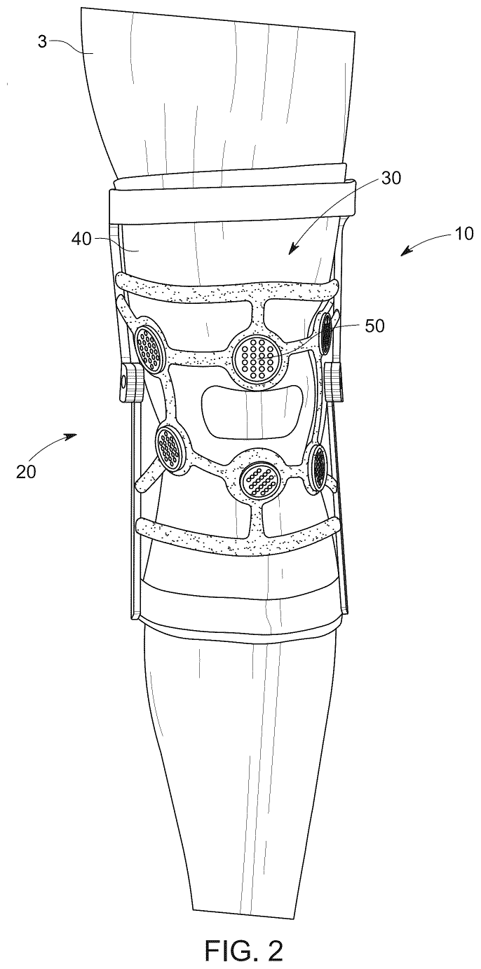

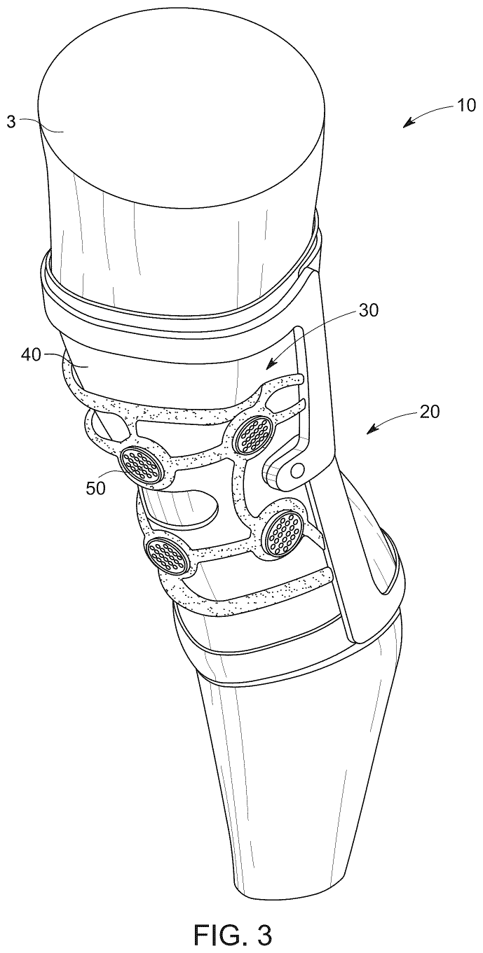

General aspects of this disclosure are now described with reference to a muscoskeletal apparatus 10. As shown in FIGS. 1-3, muscoskeletal apparatus 10 may be wearable by a user 1 to support their muscoskeletal system, administer “energy prescriptions”, and output sensory data associated with user 1's usage of muscoskeletal apparatus 10 and the efficacy of any energy prescriptions administered to user 1 with apparatus 10.

Muscoskeletal apparatus 10 may support any portion of user 1's muscoskeletal system, including any joint of any limb or digit. As shown in FIG. 1, apparatus 10 may support a knee 5 of a leg 3 of user 1 during bending movements of leg 3 like those experience in the normal course of daily activities. Muscoskeletal apparatus 10 also may maintain a position of one or more, or a plurality of multi-energy generators at a treatment location(s) on leg 3 during the movements thereof, allowing apparatus 10 to administer energy prescriptions over extended periods of time, including times when user 1 is the on the move. Apparatus 10 may provide a communication platform for capturing, outputting, and inputting multiple forms of sensory data, making it possible for a prescriber of an energy prescription (e.g., a medical doctor) to monitor both user 1's usage of apparatus 10 as a support device and indicators associated with the efficacy of any energy prescriptions administered with apparatus 10.

As utilized herein, an “energy prescription” may comprise a quantifiable dose of a first energy-based treatment output toward skin 2 of user 1 by itself or with a quantifiable dose of a second treatment. The first energy based treatment may comprise any combination of one or more different energy types output toward the skin, such as a combination of an electrical energy, a light energy, a thermal energy (hot or cold), a pressure energy, a vibratory energy, and/or any other type of energy output from an electromechanical generator element. Any type of second treatment may be used in combination with the first treatment to provide user 1 with a combined and/or sustained benefit that otherwise may not be obtainable on its own. For example, the second treatment may comprise any one or more of: (a) a biologic-based treatment delivered by injection through skin 2; (b) a chemical-based treatment delivered by injection through or application to skin 2; (c) a second energy-based treatment output to skin 2, such as a second combination of an electrical energy, a light energy, a thermal energy, a pressure energy, vibratory energy, and/or the like; and/or (d) a pharmacological-based treatment that is consumed by user 1, delivered by injection through or application to skin 2, or otherwise administered to user 1.

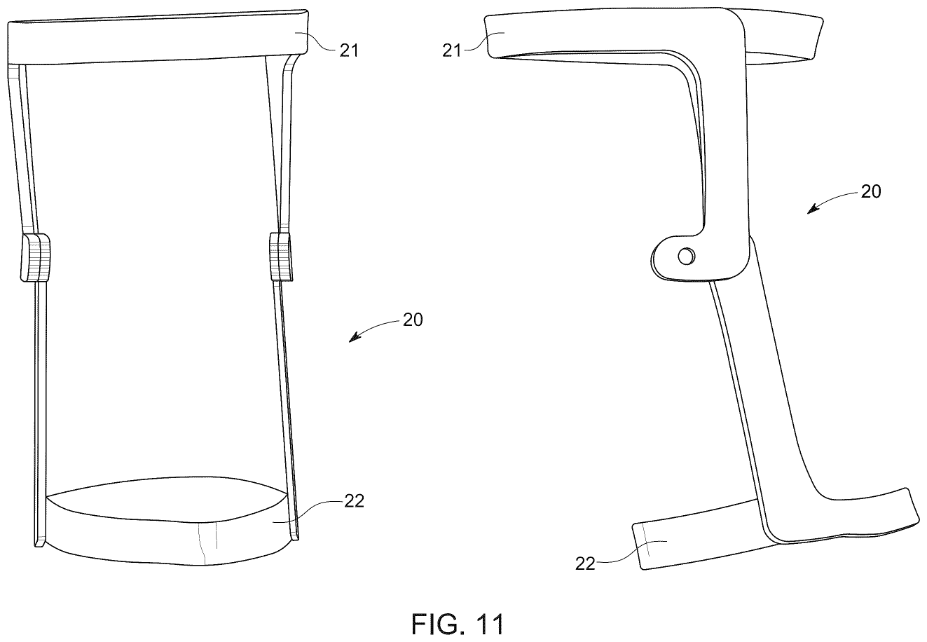

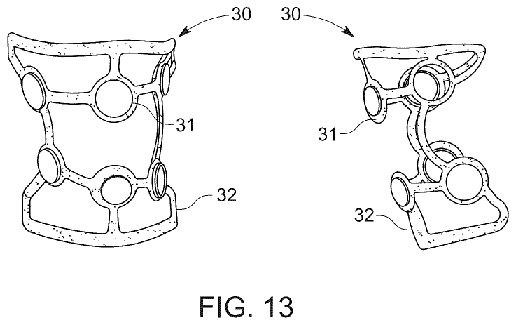

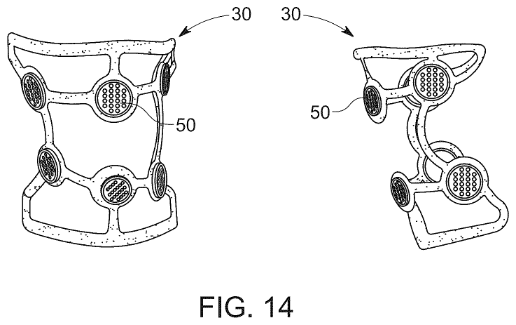

Particular aspects of this disclosure are now described with continued reference to a muscoskeletal apparatus 10 configured to be worn on leg 3, although many aspects may generally be applicable to and similarly described in relation to another joint of another limb or digit of user 1. As shown in FIGS. 1-15, muscoskeletal apparatus 10 may comprise a brace 20, a lattice structure 30, a sleeve 40, and a multi-energy generator 50 operable to output an energy treatment toward a treatment area of leg 3 when apparatus 10 is worn by user 1.

Brace 20 may comprise rigid or semi-rigid bracing structures that are pivotable about and relative to a rotational axis of knee 5 during movements of leg 3. The bracing structures may guide and support movements of leg 3. For example, brace 20 may be wearable to guide and support knee 5 by limiting first movements of superior portion of leg 3 relative to an inferior portion of leg 3 (e.g., including a foot 4) in an anterior-posterior direction to prevent or treat a hyperextension of knee 5 and limiting second movements of the superior portion relative to the inferior portion in one or more medial-lateral directions to prevent or treat injuries to the ACL or MCL of knee 5. Unlike a traditional knee brace, brace 20 may comprise one or more multi-energy generators 50 operable apply energy-based treatments to knee 5 when apparatus 10 is worn by the user. As described herein, muscoskeletal apparatus 10 may comprise one or more, or a plurality of multi-energy generator(s) 50 operable to apply energy-based treatments to a corresponding one or more, or a plurality of treatment site(s) of knee 5.

As shown in FIGS. 1-3 and/or 12, brace 20 may comprise a superior frame 21, an inferior frame 22, and a hinge system 23.

Superior frame 21 may be wearable above knee 5 on a superior portion of leg 3. As shown in FIGS. 1-3 and/or 11, superior frame 21 may comprise a beam 24 with a 3D curved shape that wraps around a superior curved portion of leg 3 and an attachment 25 operable to maintain an interior, skin-facing surface of beam 24 against an exterior surface of the superior curved portion of leg 3. For example, beam 24 may be laser cut from a flat sheet of aluminum according to a 2D plan sheet and bent into the depicted 3D curved shape by man or machine; and attachment 25 may comprise a superior tensioning system comprising a strap with Velcro® attachments.

Inferior frame 22 may be wearable below knee 5 on an inferior portion of leg 3. As shown in FIGS. 1-3 and/or 11, inferior frame 22 may comprise a beam 26 with a 3D curved shape that wraps around an inferior curved portion of leg 3 (e.g., at a location below knee 5 and above foot 4) and an attachment 27 operable to maintain an interior, skin-facing surface of beam 26 against an exterior surface of the inferior curved portion of leg 3. Continuing the previous example, beam 26 may laser cut from the same flat sheet of aluminum and similarly bent into the depicted 3D curved shape by man or machine; and attachment 27 may comprise an inferior tensioning system comprising another strap with Velcro® attachments.

Hinge system 23 may comprise a lateral hinge operable with a medial hinge to limit the first movements of superior frame 21 relative to inferior frame 22 in an anterior-posterior direction and the second movements of frame 21 relative to frame 22 in the one or more medial-lateral directions. As shown in FIGS. 1-3 and/or 11, hinge system 23 may comprise anterior stop surfaces of superior frame 21, posterior stop surfaces of interior frame inferior frame 22, and a pin that rotatably couples frame 21 with frame 22 so that the first movements of superior frame 21 relative to inferior frame 22 in the anterior-posterior direction are limited when the anterior stop surfaces contact the posterior stop surfaces. As further shown in FIGS. 1-3 and/or 11, hinge system 23 may comprise interior guide surfaces of superior frame 21 that are rotatably coupled to exterior guide surfaces of interior frame inferior frame 22 with the pin so that the second movements of frame 21 relative to frame 22 in the medial-lateral direction(s) are limited when the interior guide surfaces slide past the exterior guide surfaces.

Brace 20 may thus be used to support and promote healing of knee 5 by (i) limiting the first movements of superior frame 21 and leg 3 above knee 5 relative to relative to inferior frame 22 and leg 3 below the knee in the anterior-posterior direction (ii) while simultaneously and dynamically limiting the second movements of superior frame 21 and leg 3 above knee 5 relative to relative to inferior frame 22 and leg 3 below the knee in the one or more medial-lateral directions. Hinge system 23 may be modified to accommodate different movements of leg 3. For example, hinge system 23 may thus comprise any combination of bi-directional and/or omni-directional hinges that are located on a medial and/or lateral side of the knee and operable to limit movements of superior frame 21 relative to inferior frame 22. By way of example, hinge system 23 may comprise any structures like those described in U.S. Pat. Nos. 5,415,625; 7,794,416; or 10,485,689, the entireties of which are hereby incorporated by reference into this disclosure.

Beams 24, 26 may comprise rectangular cross-sections that are shaped and sized to resist deformation of their 3D curved shapes responsive to reaction forces generated when moving leg 3 during normal use, such as when user moves leg 3 to run and/or experiences external impact forces while running. The structural and material composition of brace 20 may be optimized to (a) maintain the structure integrity of apparatus 10 by resisting deformations of frames 21, 22; (b) serve as a heat sink for multi-energy generator 50 by enhancing convection; and (c) provide attachments for other elements of apparatus 10 (e.g., lattice structure 30) and electronic components operable with multi-energy generator 50 (e.g., a battery). Beams 24, 26 may be made of any material or composite material strong enough to resist deformation. All or parts of brace 20, such as superior frame 21, inferior frame 22, and/or hinge system 23, may be made of a metallic material (e.g., aluminum) and/or a biocompatible composite material (e.g., such as acrylonitrile butadiene styrene or “ABS”) with a metallic core (e.g., an aluminum core). Although not required unless claimed, aluminum may be a good choice for beams 24, 26 because it is lightweight, has a high thermal conductivity, has a low electrical conductivity, and is biocompatible.

Lattice structure 30 may define an elastic open framework extending around all or portion of knee 5 when apparatus 10 is worn, such as when the superior and inferior portions of leg 3 are attached to respective superior and inferior portions of brace 20. All or portions of the open framework may be formed or printed with a biocompatible elastic material (e.g., such as nylon, silicone, and the like) that is wearable on or adjacent knee 5 for extended periods of time. An elasticity of the elastic open framework may allow lattice structure 30 to undergo a repeatable set of deformation stresses when user 1 dons, wears, and removes apparatus 10. For example, when user 1 dons the apparatus 10 by attaching portions of brace 20 to portions of leg 3, portions of the lattice structure 30 may expand initially to receive knee 5, allowing structure 30 to be fit over knee 5, and then elastically contract obtain around knee 5 when moving by exerting forces that press structure 30 against skin 2.

As shown in FIGS. 1-3 and 14-15, lattice structure 30 may comprise a plurality of nodes 31, a plurality of cords 32, an electrical network 33, and a thermal network 34. Plurality of cords 32 may comprise an elastic open framework that structurally interconnects plurality of nodes 31 with one another and/or brace 20, and provides passageways for wires extending between nodes 31 and/or brace 20. Some cords 32 may be span between one or more nodes 31 opposing portions of brace 20, such as between medial and lateral surfaces of frames 21, 22, so that lattice structure 30 expands and contracts to continually press plurality of nodes 31 toward skin 2 when apparatus 20 is worn. As shown in FIGS. 1-3 and 13-14, lattice structure 30 may elastically conform around knee 5 of leg 3 when apparatus 10 is worn by the user and remain conformed around knee 5 when user 1 moves leg 3 by resiliently expanding or contracting in different directions when brace 20 is straight or bent.

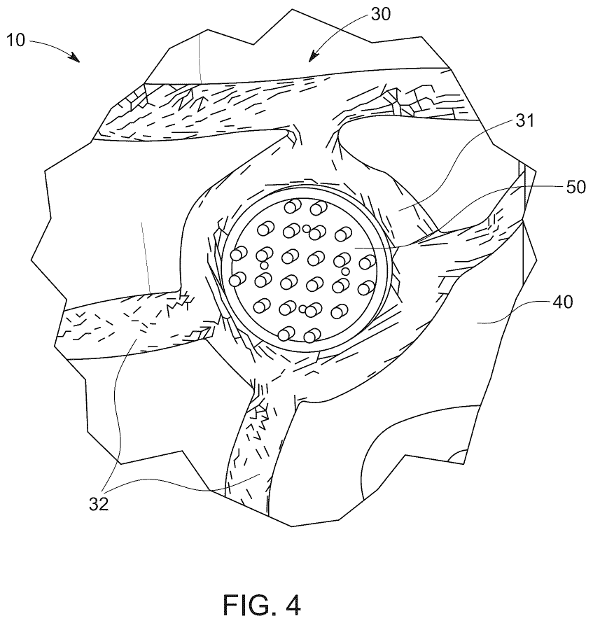

Each node of plurality of node 31 may contain a data communication device and be operable with one or more cords of plurality of cords 32 to maintain a position of that device relative to a treatment site on knee 5. The plurality of communication devices may contain any combination of one or more single-or multi-energy generators, sensors, controllers, processors, transceivers, power sources, and/or like components. By way of example, each node 31 is shown in FIGS. 4-7 as containing one multi-energy generator 50 as described further below. Other devices may be similarly deployed, including any of data communication device described in U.S. patent Ser. No. 17/797,361, the entirety of is incorporated into this disclosure.

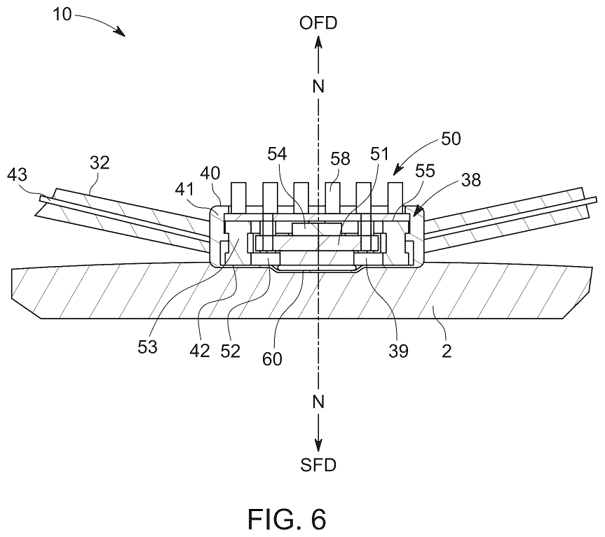

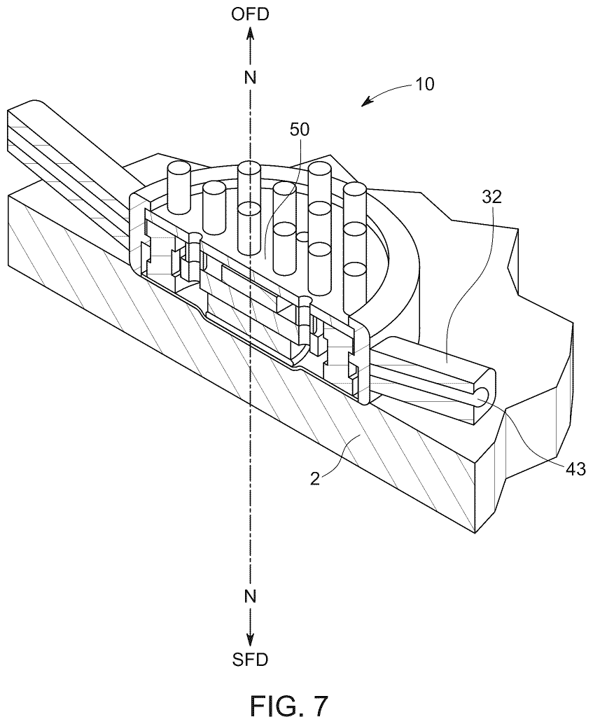

An exemplary cross-section of one node 31 is shown in FIGS. 4-7 as comprising a body 38 and a bay 39. As shown in FIGS. 6-7, body 38 may extend along a node axis N-N to define an outward-facing surface 40, a sidewall 41, and a skin-facing surface 42. Bay 39 may comprise a cylindrical container extend partially through body 38 along node axis N-N so outward-facing surface 40 and sidewall 41 comprise an annular shape with a central opening extending along node axis N-N. Sidewall 41 may be described as an annular surface of revolution about node axis N-N with a thickness sized to limit any deformations of body 38 that might cause multi-energy generator 50 to fall out when apparatus 10 is worn. As shown in FIGS. 6-7, interior surfaces of side wall 41 may have insets and protrusions defining grooves that are part of the annular surface of revolution and thus coaxial with node axis N-N.

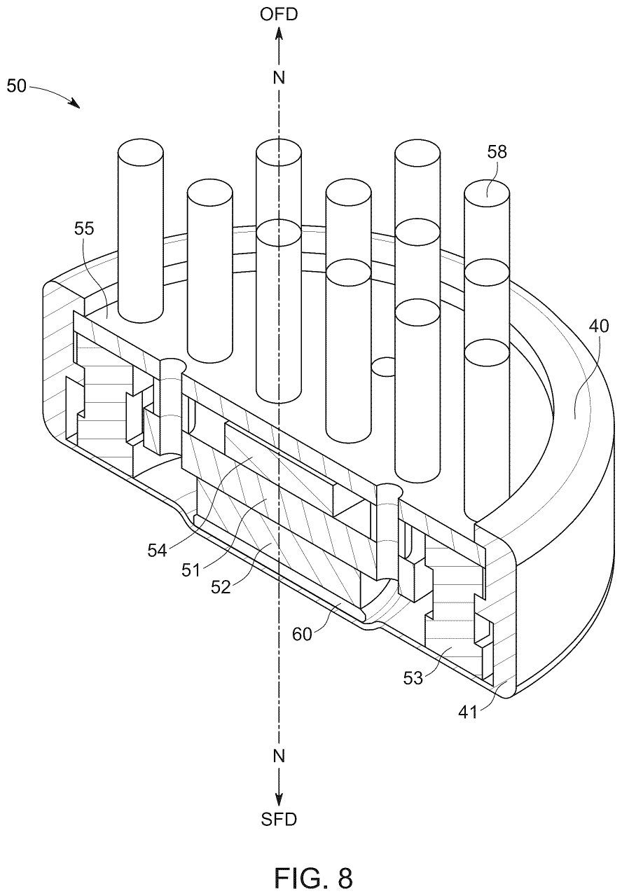

As shown in FIGS. 7-8, skin-facing surface 42 may comprise thin layer of biocompatible material that seals bay 39 at a skin-facing end of body 38. Skin-facing surface 42 may be conformable against a curved shape of leg 3. Skin-facing surface 42 may comprise material characteristics, geometrical features, and/or surface treatments operable to maintain a minimum coefficient of friction with skin of leg 3 in a variety of usage conditions, such as when apparatus 10 is worn dry, exposed to heat and/or perspiration, and when partially or fully submerged in water. Skin-facing surface 42 may, comprise a medical grade silicone having a minimum coefficient of friction with skin 2 of approximately (0.61+/−0.21)¹. To enhance its frictional engagement with skin 2, skin-facing surface 42 may comprise localized geometrical features that intentionally roughen and/or otherwise increase its surface area. Portions of the skin-facing surface 42 may be covered with a mild biocompatible adhesive or tacky material. A size and/or shape of skin-facing surface 42 may be maximized, increased, and/or otherwise modified to increase a probability that at least a portion of surface 41 will be generally and consistently maintained against skin 2 during vigorous movements of leg 3, such as when apparatus 10 is worn while playing sports. As a further example, skin-facing surface 42 may comprise a flexible concave shape (e.g., like a suction cup) that may be pressed against the skin of leg 3 to form a sealing edge therewith.

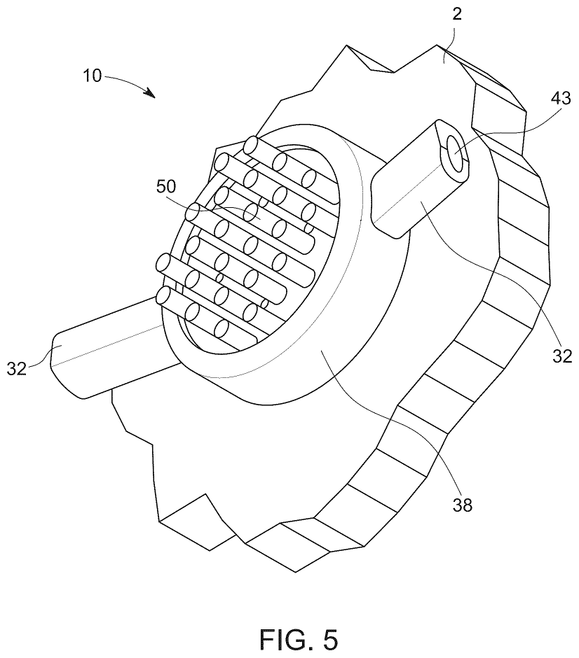

As shown in FIGS. 4-7, each sidewall 41 of each node 31 may be engaged to one or more cords 32 and adapted to transfer tensile forces back to brace 20 when apparatus 10 is worn. Each sidewall 41 of each node 31 may be structurally interconnected to at least one of another sidewalls 41 of another node 31 or brace 20 by at least one cord 32 extending therebetween. As shown in FIGS. 5-7, each cord 32 may comprise a sidewall defining generally circular cross-sectional shape with an interior lumen 43 extending between and through two different body sidewalls 42 of two different nodes 31. Interior lumens 43 may extend completely or partially through sidewalls 42. Opposing ends of each cord 32 may engage to two different sidewalls 42 and/or one sidewall 41 and brace 20 so that at least a central portion of each cord 32 may elastically expand and contract (e.g., like a spring) when user 1 dons, wears, and removes apparatus 20. The material characteristics and/or thicknesses of cords 32 and sidewalls 42 may define elastic properties of each cord 32, such as a spring constant of its central portion and/or a maximum amount of elastic force applicable therewith.

Each body 38 of each node 31 may thus contain one multi-energy generator 50, maintain its position relative to knee 5, and direct its energies toward skin 2. As shown in FIGS. 7-8, the one multi-energy generator 50 may be inserted into bay 39 in either a skin-facing direction or “SFD” along node axis N-N or an outward-facing direction or “ODF” along node axis N-N.

As shown in FIGS. 6-10, each multi-energy generator 50 may comprise a plurality of generator elements (e.g., elements 52, 53 below) operable to output a plurality of different energy types in a signal direction toward a treatment site on leg 3 when muscoskeletal apparatus 10 is worn by user 1. Because of its multi-energy capabilities, including its ability to deliver precise amounts of energies to strategic locations relative to portions of the user's muscoskeletal system, each energy generator 50 of apparatus 10 may be operable to administer a plurality of different energy prescriptions to user 1 over multiple treatment periods by causing one or more generator elements of the plurality of generator elements to output different energy signals during each treatment period. Each energy signal may comprise a different combination of electrical energy, light energy, thermal energy, pressure energy, and/or vibratory energy, depending upon the configuration and capabilities of the plurality of generator elements.

Because each generator element is an electro-mechanical device, multi-energy generator 50 may be operable output precise amounts of energies, allowing generator 50 to administer quantifiable amounts or “doses” of an energy prescription by itself, such as by outputting a series of different energy signals; or in combination with any one or more of a biologic-based treatment, a chemical-based treatment, and/or a pharmacological-based treatment, such as by outputting energy signals optimized for use with such treatments. Put another way, according to this disclosure, each generator element of the plurality of generator elements may be independently operable to output one energy type of the plurality of different energy types in a skin-facing direction (or “SFD”); and energy generator 50 may be operable to cause one or more of the generator elements to administer an energy prescription by outputting an energy signal in the signal direction with one or more energy types of the plurality of different energy types when muscoskeletal apparatus 10 is worn by user 1, including times when user 1 is stationary or moving.

A cross-section of an exemplary data communication device is shown in FIGS. 7-10 and now described with continued reference to multi-energy generator 50. As shown, multi-energy generator 50 may comprise a printed circuit board or “PCB” 51, a first generator element 52, at least one second generator element 53, a controller 54, a backing plate 55, and a heat sink 56.

PCB 51 may electrical and structurally connect first generator element 52, at least one second generator element 53, and controller 54 to one another, forming a “PCB Assembly”. PCB 51 also may structurally connect the PCB Assembly to backing plate 55. PCB 51 may comprise any type of non-substrate materials (e.g., aluminum, glass, or resin) and/or conductive layer(s) (e.g., copper circuitry) extending between different mounting pads for first generator element 52, second generator element(s) 53, and controller 54. For example, like an LED, PCB 51 may comprise an aluminum non-substrate material offering superior heat dissipation and transfer to backing plate 55.

First generator element 52 may output a first energy type of the plurality of different energy types in the SFD for multi-energy generator 50. As shown in FIGS. 7-10, first generator element 52 may comprise an actuator that is connected to a skin-facing side of PCB 51 and operable to output a vibrational energy in the signal direction. For example, first generator element 52 may comprise a linear resonate actuator (or “LRA”) operable to output a first range of vibrational frequencies and/or a piezoelectric actuator operable to output a second range of vibrational frequencies, such as one that is wider than the first round. By way of example, the LRA may have an operating side mounted to a pad of PCB 51 and an energy outputting side adjacent an outward-facing side of skin-facing surface 42. As a further example, exterior side surfaces of the LRA may be spaced apart from interior surfaces of second generator element 53 to define a thermal break (e.g., an air gap) therebetween; and the thermal break may be filled with an epoxy or other thermally insulating material.

Second generator element 53 may output a second energy type of the plurality of different energy types in the SFD. Multi-energy generator 50 may comprise any number of second generator elements 53, including combinations and/or configurations described in one or more of U.S. Pat. No. 10,959,674 and its progeny, U.S. patent Ser. No. 17/797,361 and its progeny, U.S. patent Ser. No. 18/024,371 and its progeny, U.S. patent Ser. No. 17/922,791 at its progeny, U.S. Prov. Pat. App. No. 63/414,140 and its progeny, and U.S. Prov. Pat. App. No. 63/356,950 and its progeny, the entireties of which are hereby incorporated into this disclosure. For example, in keeping with the incorporated references, first generator element 52 may be operable to output a first energy type of the plurality of different energy types in the signal direction; and second generator element 53 may be operable to output a second energy type of the plurality of different energy types in at least one of: the signal direction and a plurality of different signal directions relative to the signal direction.

Second generator element 53 may at least partially surround first generator element 52 so that the first energy type and the second energy type are output in the signal direction toward a similar treatment area of knee 5, allowing for combinatory treatments that are only applicable when both of the first and second energy types are output with multi-energy generator 50. By way of example, as shown in FIGS. 8-10, second generator element 53 may comprise a thermoelectric cooler module (or “TEC Module”) with an annular perimeter shape that surrounds first generator 52 and a I-shaped cross-section similar to that of a wide-flanged I-beam.

An outward-facing flange of the TEC Module (e.g., a first aluminum ring) may be attached to a skin-facing side of backing plate 55 and a skin-facing flange of the TEC Module (e.g., a second aluminum ring) may be attached to an outward facing side of skin-facing surface 42 of body 38. The TEC Module may comprise an annular array of Peltier coolers that are located between its outward-facing and skin-facing flanges and electrically connected to PCB 51 when forming the PCB Assembly.

As shown in FIGS. 8-10, when the PCB Assembly is formed, interior surfaces of the TEC Module between its outward-facing and skin-facing flanges and the annular array of Peltier coolers located therebetween may define an interior indention. An edge of PCB 51 may be received in the interior indention and spaced apart therefrom to define a thermal break (e.g., an air gap) therebetween. The resulting thermal break may be filled with an epoxy or other thermally insulating material. As further shown in FIGS. 8-10, exterior surfaces of the TEC Module between its outward-facing and skin-facing flanges and the annular array of Peltier coolers located therebetween may define an exterior indention and a protrusion of sidewall 41 may be received in the exterior indention to snap-fit the PCB Assembly into bay 39 and deformed to remove the PCB Assembly therefrom. The skin-facing flange of the TEC Module may be pressed against the outward-facing surface of skin-facing surface 42 of body 38 when the PCB Assembly is snap-fit into bay 39. As shown in FIG. 8, a force-transmitting nub 60 may be placed in contact with first energy generator 51, such as between the exemplary LRA and skin-facing surface 42. Nub 60 may comprise a vibrational force amplifying material that is denser than skin-facing surface 42 and has a surface area larger than that of the LRA, allow it to more efficiently distribute vibrational forces to a discrete area of skin 2. By way of example, nub 60 may comprise a thickened portion of surface 42 that is sized for receipt inside the skin-facing flange of the TEC Module.

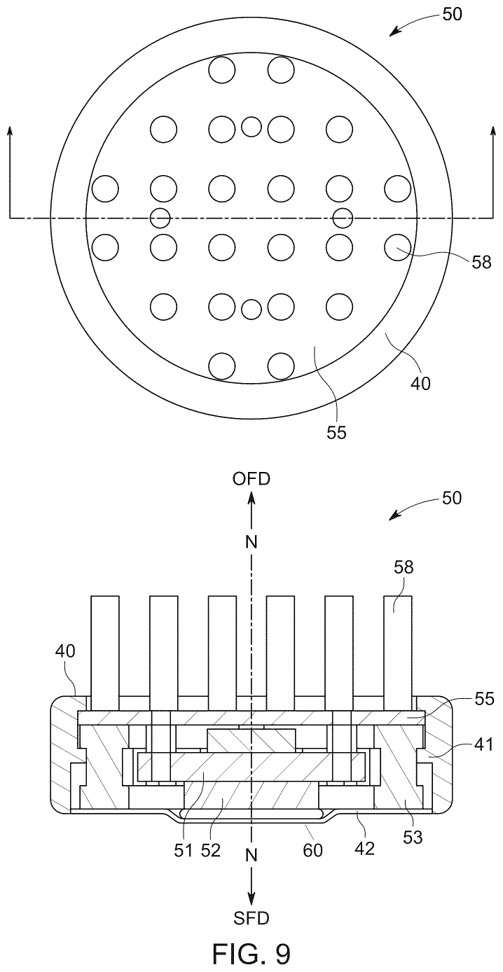

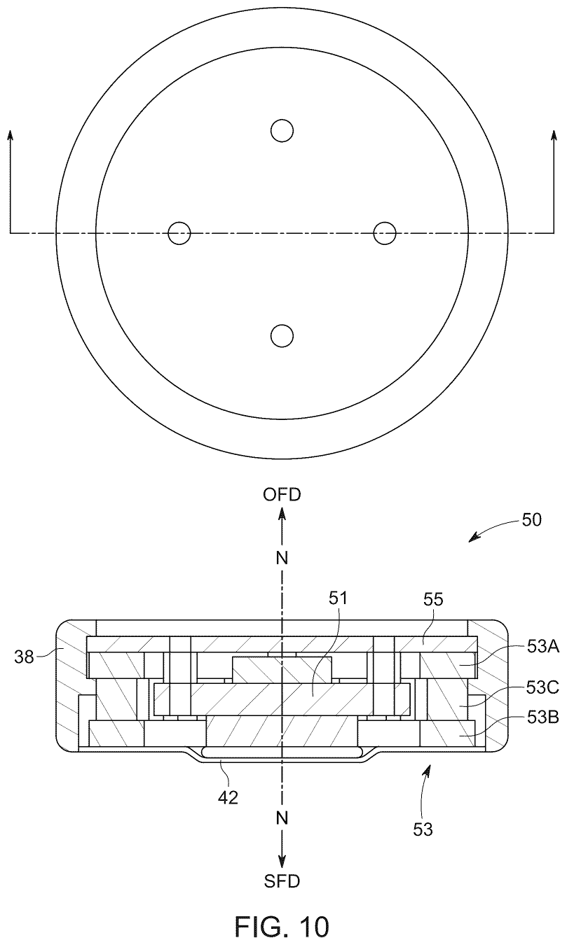

Controller 54 may be operable to cause one or more generator elements of the plurality of generator elements of multi-energy generator 50 to output an energy signal in the signal direction with one or more energy types of the plurality of different energy types when the apparatus 10 worn. As shown in FIGS. 9-10, a skin-facing side of controller 54 may be operatively connected to an outward-facing side of PCB 51 so that the controlling elements of multi-energy generator 50 are spaced apart from skin-facing surface 42 and any moisture leaks associated therewith. Controller 54 may comprise a microcontroller and related components for distributing power to the plurality of generator elements of multi-energy generator 50, such as to first generator element 52 and second generator element 53. As shown in FIGS. 9-10, an outward-facing side of controller 54 may be thermally connected to the skin-facing side of backing plate 55 and operable to discharge excess heat thereto.

In keeping with previous examples, controller 54 may comprise circuitry and drivers operable to cause the skin-facing flange of the TEC Module output either a hot thermal energy or a cold thermal in the SFD. Because they are Peltier coolers, the respective outward-facing and skin-facing flanges of the TEC Modules will operate equally and oppositely. For example, if the skin-facing flange of the TEC Module is heated by plus 10-50 degrees then the superior, outfacing flange will be cooled by negative 10-50 degrees. Larger temperature swings can create problems with excess heat, such as when cooling the inferior, skin facing surface to minus 50 degrees heats the superior, skin facing to plus 50 degrees or higher when combined with body heat output from leg 3. Aspects of heat sink 56 may be operable to help discharge the excess heat when this happens, making it easier to cycle multi-energy generator 50 between different hot cold cycles for extended periods of time. As shown in FIGS. 7-9, connecting a middle portion of the TEC Module to an edge of PCB 51 may help with transferring excess heat to heat sink 56 because the middle portion of a Peltier cooler does not experience the same type of thermal changes as its interior and superior surfaces, meaning that less heat with be transferred to PCB 51 during operation of the TEC Module.

As shown in FIGS. 7-10, depending upon the quantify of excess heat to be transferred, heat sink 56 may comprise backing plate 55 (e.g., as shown in FIG. 10) by itself and/or a version of backing plate 55 including a plurality of fins 58 extending outwardly therefrom (e.g., as shown in FIGS. 7-9). Backing plate 55 may be made of any material (e.g., like aluminum) that attracts excess heat from second generator element 53 and dissipates it by convention to the surrounding air via an outward-facing surface area of backing plate 55 and/or an even larger surface provided by plurality of fins 58. The middle-to-edge connection of the TEC Module to PCB 51 and heat sink 56 thereby provides steady state solutions for helping multi-energy generator 50 to dissipate excess heat, meaning apparatus 50 does not require any moving parts to output thermal energies of plus/minus 50 degrees in the SFD, such as an electrical fan.

Multi-energy generator 50 may not comprise a housing per se. Instead, backing plate 55 may be operable with skin-facing surface 42 to structural attach the aforementioned PCB Assembly to node 31 and seal it in bay 39, such that body 38 is the housing. As shown in FIGS. 8-10, the PCB Assembly may be attached to a skin-facing side of backing plate 55 by one or more elongated rods or “standoffs” extending therebetween. Each standoff may extend from PCB 51, through an opening in backing plate 55, and be attached to plate 55 adjacent the opening (e.g., via an adhesive or weld).

Alternatively, in keeping with FIG. 8, body 38 may comprise a housing for multi-energy generator 50, making it a standalone device. For example, skin-facing surface 42 may comprise a biocompatible adhesive operable to maintain a position of energy generator 50 relative to user 1 by adhering body 38 to skin 2. In this example, controller 54 may comprise a power source (e.g., such as a battery and/or a coil operable for wireless power transfer) and a data transceiver (e.g., such as a Bluetooth or WIFI chip), allowing plurality of generators 50 to communicate wirelessly.

As shown in FIGS. 1-3, lattice structure 30 may comprise an elastic portion of apparatus 10 that is structurally attached to brace 20 and operable therewith to press skin-facing surfaces 42 of nodes 31 against skin 2. Each cord 32 may comprise an elastic cord portion, such as an outer biocompatible layer; and a metallic portion, such as wires located inside the outer layer. The elastic cord portions may be tensioned between the elements of brace 20 so that they continuously press skin-facing surfaces 42 of nodes 31 toward different treatment sites of skin 2 during the aforementioned first and second movements of frames 21, 22. As shown in FIG. 1, some of wires may be structurally and thermally connected to beams 24, 26 of frame 20 so that frames 24, 26 become operable to discharge excess heat from multi-energy generator 50, preventing it from over-heating or over-cooling without using an electric fan.

Electrical network 33 may comprise electrical conductors (e.g., copper wires that are part of the aforementioned wires) that extend through lumens 43 of cords 32 between each multi-energy generator 50 and are operable to transmit power and/or data between generators 50 and/or other elements associated therewith, such as a power source for apparatus 10, such as rechargeable battery removably attached to brace 20, such as a rechargeable lithium ion battery. As shown in FIG. 1, for example, beams 24, 26 of frame 20 may comprise hollow portions with interior cavities sized to receive one or more rechargeable batteries sized for receipt inside beams 24, 26. The shape, size, and position of the batteries may be optimized so that their weight is evenly distribution on both sides of apparatus 10, extending its run time.

Frame 20 may be formed from aluminum or 3D printed from a metallic with heat transfer properties like those of aluminum, allowing beams 24, 26 to more effectively discharge heat generated by the power source and function as part of thermal network 34. As shown in FIG. 1, thermal network 34 also may comprise thermally insulated thermal conductors (e.g., thermally insulated aluminum wires or rods that are part of the aforementioned wires inside cords 32) that extend through lumens 43 cords 32 between each multi-energy generator 50 beams 24, 26 of brace 20, making them operable to transmit thermal energies from backing plates 55 to beams 24, 26 of brace 20, and therebetween.

As shown in FIGS. 1-3, sleeve 40 may comprise a biocompatible elastic structure that is wearable directly against skin of the muscoskeletal joint for comfort and support. Sleeve 40 may extend along a longitudinal axis of leg 3. As shown in FIGS. 1-3, sleeve 40 may extend along the longitudinal axis of a leg of user 1 with a superior portion extending above the knee 5 and an inferior portion below the knee to define a covered portion of leg 3 that partially encloses the knee. Sleeve 40 may comprise a thermal management material selected for a heating or cooling treatment. For heating applications, sleeve 40 may comprise a heat reflective layer (e.g., a metallic layer) operable to reflect heat generated by leg 3 and/or heat generated by plurality of generators 50 back toward leg 3 in order to maintain a heated or above-normal temperature of the knee. Conversely, for cooling applications, sleeve 40 may comprise perforations, channels, fins, or like structures operable to transfer heat generated by leg 3 and heat generated by plurality of generators 50 into the surrounding air in order to maintain a cooled or below-normal temperature of the knee.

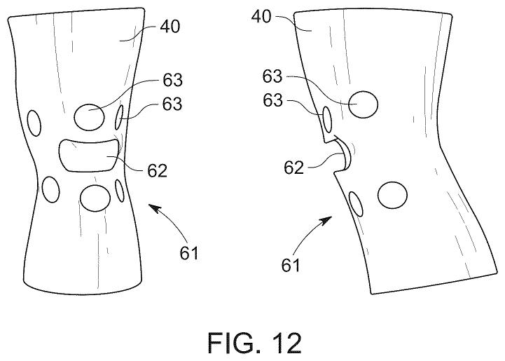

As shown in FIG. 12, sleeve 40 may comprise a plurality of openings 61 including an injection opening 62 and an interface opening 63 for each multi-energy generator 50. Injection opening 62 may be sized to permit injection of a treatment fluid into the knee of user 1 without removing apparatus 10. As shown in in FIG. 12, injection opening 62 may be located above a patella of the knee because that is a common injection site for many treatment fluids. The location and number of injection openings 62 of sleeve may be modified as needed to accommodate different injection sites. For example, sleeve 41 may comprise a plurality of openings 61 like that shown in FIG. 12 and/or portions of sleeve 40 may be loosely woven to permit injections of the treatment fluid between warps and wefts of sleeve 40. Sleeve 40 may be worn for hours or days at time and be optimized for long-term wearability. For example, sleeve 40 may comprise a biocompatible silicone material or exterior layer, a silver coating or threading, and/or a pharmaceutical composition or coating operable to prevent contact-based sores.

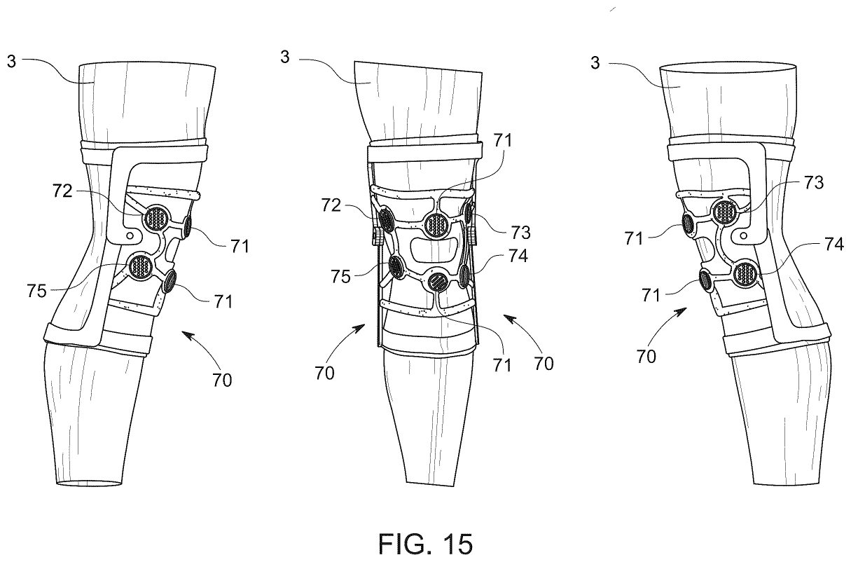

In keeping with above, by way of example, muscoskeletal apparatus 10 may be wearable to support knee 5 and comprise a plurality of multi-energy generators 50 positioned to output energy signals to a plurality of different treatment sites 70 about knee 5. As shown in FIG. 15, different treatment sites 70 may comprise one or more first sites 71 approximate to the patella (e.g., shown as above and below), a second site 72 proximate to the ACL, a third site 73 proximate to the MCL, a fourth site 74 proximate to the pes anserine, a fifth site 75 below the ACL. In this example, each energy generator 50 may be operable to administer a different portion of an energy prescription to each treatment site 70, making it possible to optimize each energy prescription for the particular needs of a particular user 1 at targeted locations about the knee.

To support measurement and verification of variables associated with usage, muscoskeletal apparatus 10 may comprise one or more usage sensors such as microphones for capturing audio data (e.g., for listening to the tissues for “pops” or grinding sounds typically associated with injury), IMUs for tracking movements leg 3, and ultrasonic transducers for measuring changes internal to knee 5. To support measurement and verification of variables associated with the energy prescription, each energy generator 50 may comprise one or more efficacy sensors operable to generate physiological data associated with each treatment site 70, such as blood flow, pH, temperature, and the like. Because of these sensors, apparatus 10 may thus output sensory data associated with user 1's usage of muscoskeletal apparatus 10 and the efficacy of any energy prescriptions administered to user 1 with apparatus 10. By way of example, the multi-energy generator 50 at the above-referenced fifth site 75 may be replaced with a sensor operable to generate data for one or more sites 70.

As described herein, muscoskeletal apparatus 10 may function as a part of a platform for data communication and energy distribution comprised of wearable components that are operable with one another to deliver energy prescriptions to user 1 and output measurement and verification data associated with the usage of apparatus 10 and an efficacy of any energy prescriptions delivered with apparatus 10.

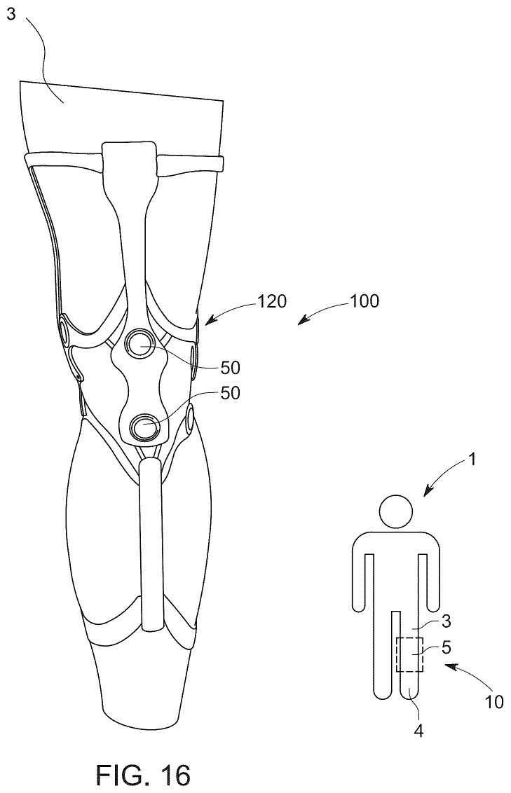

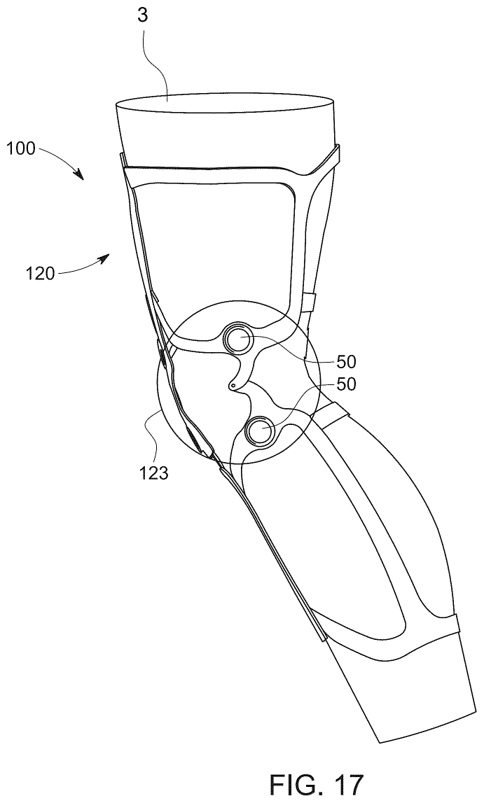

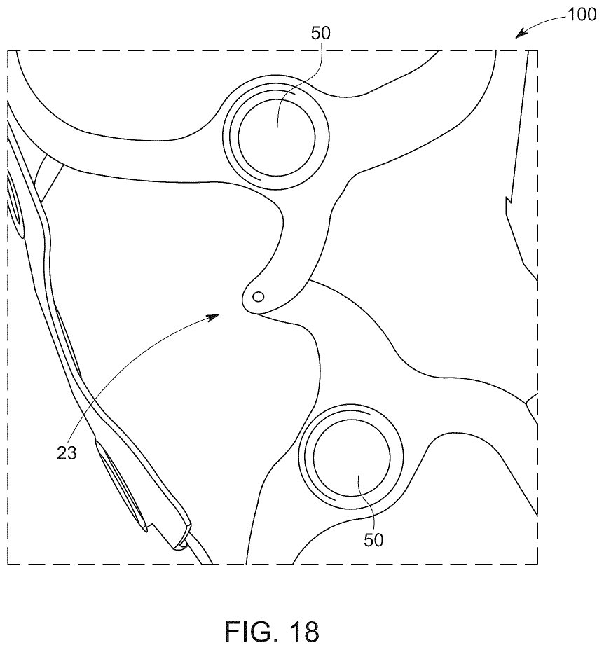

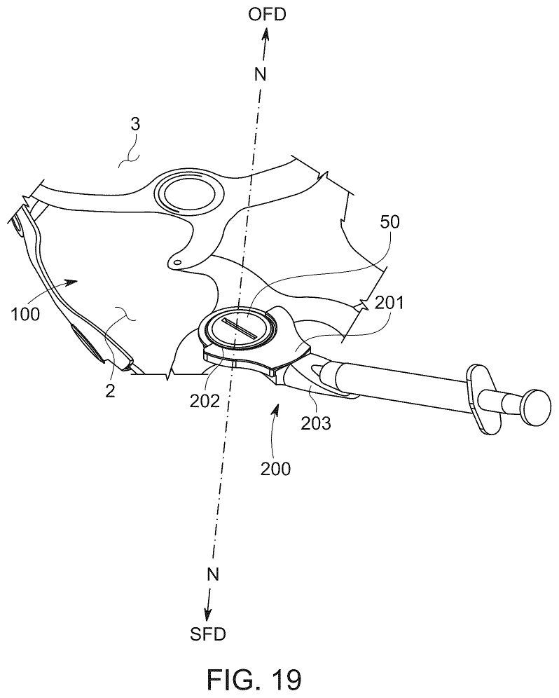

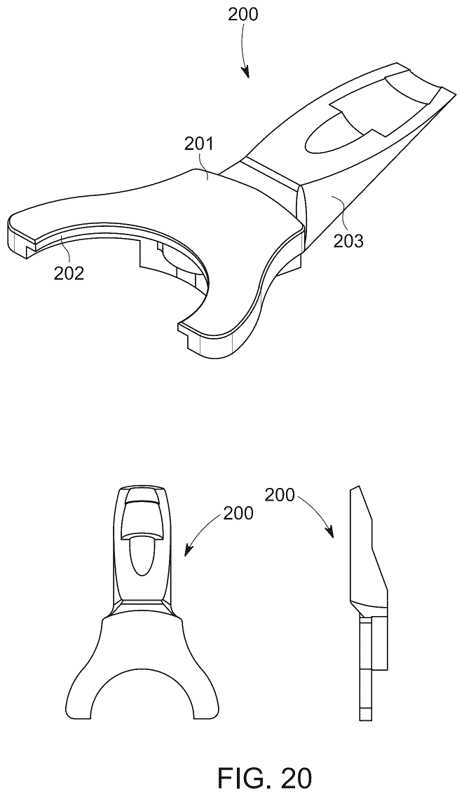

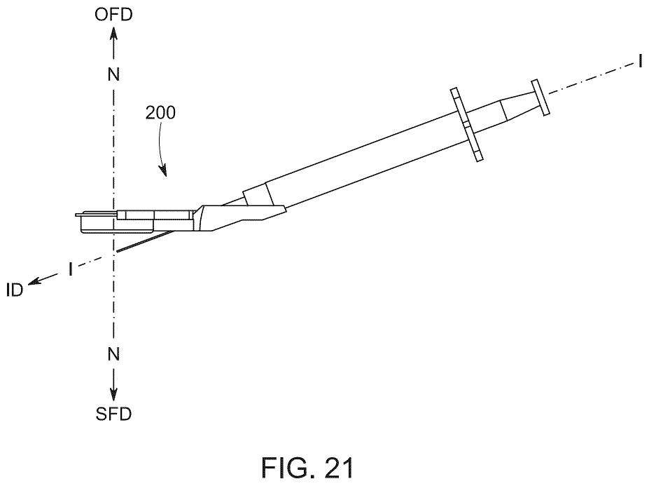

Additional aspects of this disclosure are now described with reference to: another example of muscoskeletal apparatus 10 shown in FIGS. 16-18 as a muscoskeletal apparatus 100; a treatment delivery device 200 shown in FIGS. 19-22 with reference to apparatus 100; a method 300 of administering energy prescriptions with muscoskeletal apparatus 10, 100; and a method 400 of verifying the efficacy of energy prescriptions administered with apparatus 10, 100.

Except for the differences noted below, muscoskeletal apparatus 100 may comprise elements similar to those of muscoskeletal apparatus 10, but within the 100 series of numbers, whether or not those elements are depicted in FIGS. 16-18. Any aspects described with reference to muscoskeletal apparatus 10 and/or 100 may thus be included within any variation of apparatus 10, 100 described herein, each possible combination or iteration being part of this disclosure.

In contrast to brace 20, muscoskeletal apparatus 100 may comprise a brace 120 comprising an open network of beam elements 121 operable to obtain a form-fit with leg 3. Beam elements 121 of brace 120 may define an organic exoskeleton that surrounds knee 5, providing a lighter and more flexible alternative to beams 24, 26, making it more suitable for daily use, such when used for preventative care. For example, each beam element 121 of brace 120 may comprise a thinner cross-sectional shape of a composite material (e.g., a reinforced polymeric material, like nylon) and/or flexible portions defining living hinges. Because it is form fitting, brace 120 may be more easily wearable underneath clothing and similarly operable to guide and support knee 5 by limiting first movements of superior portion of leg 3 relative to an inferior portion of leg 3 in an anterior-posterior direction to prevent or treat a hyperextension of knee 5 and limiting second movements of the superior portion relative to the inferior portion in one or more medial-lateral directions to prevent or treat injuries to the ACL or MCL of knee 5.

Brace 120 also may comprise a plurality of multi-energy generators 50 operable to apply a plurality of different energy-based treatments to a plurality of treatment sites about knee 5 when apparatus 100 is worn by user 1. As shown in FIGS. 16-18, each multi-energy generator 50 may similarly comprise a plurality of generator elements (e.g., like elements 51, 52, 53, etc. described above) operable to output a plurality of different energy types in a signal direction toward a treatment site on leg 3 when muscoskeletal apparatus 10 is worn by user 1. Because of its multi-energy capabilities, each energy generator 50 of apparatus 100 also may be similarly operable to administer a plurality of different energy prescriptions to user 1 over multiple treatment periods by causing one or more generator elements of the plurality of generator elements to output different energy signals during each treatment period.

As shown in FIGS. 19-22, treatment delivery device 200 may comprise a body 201 operable with one of nodes 31 to deliver a fluidic treatment to a location below a multi-energy energy generator 50 contained in the one node 31 so that an energy-based treatment output from generator 50 may be delivered to approximately the same spot along node axis N-N where the fluidic treatment is being delivered. As shown in FIGS. 19-22, body 201 may comprise a contact surface 202 positionable against an outer perimeter of energy generator (e.g., such as an outer perimeter of body 38) and a needle guide 203 angled to reliably guide a tip of a needle along in injection axis I-I that intersects node axis N-N at a point underneath generator 50.

Treatment delivery device 200 may be utilized to deliver different types of second treatments may be used in combination with a first treatment delivered by multi-energy generator 50, thereby providing user 1 with a combined and/or sustained benefit that otherwise may not be obtainable on its own. For example, the second treatments deliverable by treatment delivery device 200 may comprise any fluidic treatment deliverable to an interstitial space of knee 5 with a needle, such as one or more of: a biologic-based treatment like platelet-rich plasma, a chemical-based treatment such as a saline solution, and/or a pharmacological-based treatment such as an anti-inflammatory, any of which may be activated or enhanced by delivery of a second energy-based treatment output to skin 2 from generator 50, such as a second combination of an electrical energy, a light energy, a thermal energy, a pressure energy, and/or a vibratory energy.

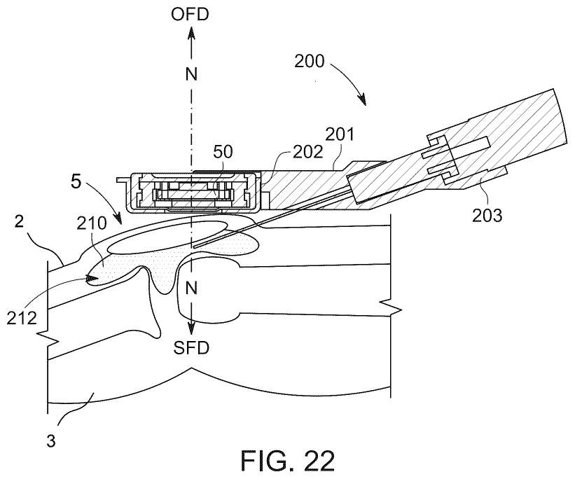

Different energies may be used to affect and/or deliver the second treatment(s) in different ways and/or at different times. As shown in FIG. 22, an exemplary fluidic treatment optimized for use with treatment delivery device 200 may comprise a biocompatible retaining gel 210 containing an energy-reactive drug delivery mechanism 211. Biocompatible gel 210 may comprise a gelatin that will remain adjacent the knee for extended periods of time after being injected into a target location for the knee, such as through injection hole 62 and/or another portion of sleeve 40 for injection adjacent or under the patella of the knee. As shown in FIG. 1, attachments 26, 27 of brace 20 may be tightened to help retain a volume of biocompatible gel 210 in the interstitial spaces surrounding the knee.

Drug delivery mechanism 211 may comprise a plurality of hydrogel microspheres 212 that were formed separately via microfluidics and cross-linking to contain separate portions of fluidic treatments. For example, hydrogel microspheres 212 may be formed by encasing a volume of a fluidic treatment in an outer shell that is disrupt able by a pre-determined output level of at least one energy type of the plurality of different energies output by multi-energy generator 50, allowing controller 54 to release the volume by causing the plurality of multi-energy generators 50 to output a plurality of different energy types in a signal direction toward the knee or another muscoskeletal joint at one treatment site of the different treatment sites, as toward the ACL or the MCL.

The plurality of hydrogel microspheres 212 may contain different portions of the fluidic treatments at one or more treatment sites for extended periods of time, making the readily available to user 1 or his attending physician. Aspects of microspheres 212 may be modified to facilitate different biological effects, therapies, and medical benefits. For example, each microsphere 212 may contain a different portion of an anti-inflammatory agent and be disrupt able when exposed to a different output level the at least one energy type, such as by having a modified outer shell made thicker and/or tougher via additional cross-linking.

To provide one example, the at least one energy type may comprise a vibratory energy signal that is output by one or more multi-energy generators 50 at certain frequencies to disrupt different percentages of hydrogel microspheres 212 via mechanical action, such as a first frequency that disrupts a first set of microspheres 212 having thinner outer shells (e.g., less than 10 nm) and a second frequency that disrupts a second set of microspheres 212 having thicker outer shells (e.g., more than 10 nm). Muscoskeletal apparatus 10 may thus be utilized to simultaneously output different quantities of the vibratory energy signal toward the knee from different directions, further ensuring that all of the microspheres 212 are exposed to a disrupting frequency.

To provide another example, the at least one energy type may comprise a thermal energy signal that is output at certain temperatures to disrupt different percentages of hydrogel microspheres 212 by chemical action. As shown in FIG. 22, because it is hydrophilic, the pH level of gel 210 may be affected by its temperature, making it possible to modify the pH level of gel 210 responsive to the thermal energy signal. The outer shells of microspheres 212 may be formed from a pH reactive gelatin that expands when exposed to higher pH levels and contracts when exposed to lower pH levels. Accordingly, when gel 210 is contained in the interstitial spaces of the knee, a first temperature below body temperature may be output to disrupt a first set of microspheres 212 by causing a pH level of gel 210 to drop below a normal measure for bodily fluids (e.g., 20° and less than 7.35-7.45) and a second temperature above body temperature may be output to disrupt a second set of microspheres 212 by causing a pH level of gel 210 to exceed the normal measure (e.g., 40° and greater than 7.35-7.45).

If the first and second sets of microspheres 212 may contain separate portions of a fluidic treatment, such as doses of pain killer, then the at least one energy type may be output at particular times via controller 54 to release additional doses of the pain killer to the treatment sites in a time-controlled manner that can be remotely accelerated or decelerated by user 1 or their attending physician. If the first and second sets of microspheres 212 contain different portions of a fluidic treatment, such as a first portion containing a pain killer and a second portion containing an anti-inflammatory agent operable therewith, then the at least one energy type may be gradually modified over time via controller 54 to release additional doses of the pain killer to the treatment sites in a time-controlled manner that can be remotely accelerated or decelerated by user 1 or their attending physician. A great variety of fluidic treatments may thus be administered in a similar time-controlled matter by muscoskeletal system 10, 100.

Related methods of administering energy prescriptions with apparatus 10, 100 may comprise steps for (1) administering a first energy-based treatment with apparatus 10, 100; and (2) administering a second treatment with apparatus 10, 100 and/or treatment delivery device 200. Related methods of verifying the efficacy of energy prescriptions with apparatus 10, 100, may further comprise, after performing method 300, additional steps for (3) gathering usage and physiological data with apparatus 10, (4) calculating an efficacy score based on the data; and (5) outputting the efficacy score to user 1 and their doctor.

While principles of the present disclosure are described herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects described herein. Accordingly, the present disclosure is not to be considered as limited by the foregoing description.

1-20. (canceled)

21. A system comprising:
a brace that is wearable on a limb, the brace comprising frames and one or more living hinges operable to guide and support movements of a muscoskeletal joint of the limb by limiting rotational movements of the muscoskeletal joint when the brace is worn;
a plurality of multi-energy generators that are attachable to the brace and positionable at different treatment sites about the muscoskeletal joint when the brace is worn, each multi-energy generator of the plurality of multi-energy generators comprising:
a plurality of generator elements operable to output a plurality of different energy types in a signal direction toward the muscoskeletal joint at one treatment site of the different treatment sites, each generator element of the plurality of generator elements being independently operable to output one energy type of the plurality of different energy types, and
a heat sink that dissipates excess heat generated from the plurality of generator elements into air surrounding the limb; and
a lattice structure comprising a plurality of cords, a plurality of nodes, and a plurality of openings,
the plurality of cords forming an elastic open framework that structurally interconnects the plurality of nodes to one another and the frame,
the plurality of nodes containing the plurality of multi-energy generators and being operable with the plurality of cords to attach the plurality of multi-energy generators to the frames and, when the brace is worn, to press the plurality of multi-energy generators toward the different treatment sites during the movements of the muscoskeletal joint,
the plurality of openings being located between the plurality of nodes.

22. The system of claim 21, wherein:
the frames comprise a first beam with a first 3D curved shape that wraps partially around a first portion of the limb on a first side of the muscoskeletal joint and a second beam with a second 3D curved shape that wraps partially around a second portion of the limb on a second side of the muscoskeletal joint; and
the one or more living hinges are operable with the first beam and the second beam to guide and support the movements of the muscoskeletal joint by limiting rotational movements of the first portion of the limb relative to the second portion of the limb during the movements.

23. The system of claim 21, wherein:
the limb is a leg;
the muscoskeletal joint is a knee of the leg comprising a patella, an ACL, an MCL, and a pes anserine; and
the different treatment sites are located approximate to the patella, the ACL, the MCL, and the pes anserine.

24. The system of claim 23, comprising a sleeve that is:
separate from the brace;
wearable under the brace; and
comprises sleeve openings that are located approximate to the different treatment sites and sized to position skin-facing sides of the plurality of multi-energy generators against skin of the muscoskeletal joint when the sleeve is worn under the brace.

25. The system of claim 24, wherein:
the sleeve openings comprise:
a plurality of interface openings sized to receive the skin-contacting surfaces of the plurality of multi-generators; and
at least one injection opening that is located to permit injection of a fluid through the sleeve and into the patella, the ACL, the MCL, or the pes anserine when the brace is worn.

26. The system of claim 21, wherein:
each heat sink comprises a backing plate and a plurality of fins extending outwardly from the backing plate; and
each multi-energy generator of the plurality of multi-energy generators is thermally coupled to the backing plate.

27. The system of claim 26, wherein:
the plurality of different energy types comprise a thermal energy comprising hot or cold; and
the plurality of generator elements of each multi-energy generator of the plurality of multi-energy generators comprise a thermoelectric device with:
a skin-facing side that is positioned on skin of the muscoskeletal joint and operable to output one of the hot or cold to the skin when the brace is worn; and
an outward-facing side that is thermally coupled to the backing plate and operable to dissipate the other one of the hot or cold into the air surrounding the limb via a surface area of the plurality of fins.

28. The system of claim 27, wherein:
the thermoelectric device comprises an annular perimeter shape and an I-shaped cross section defining an interior indention; and
a perimeter edge of the PCB is located within the interior indention and thermally isolated from the thermoelectric device with an epoxy.

29. The system of claim 28, wherein the plurality of cords define lumens extending between the plurality of the nodes and the frame and the system comprises:
electrical conductors that extend through the lumens and are electrically connected to the PCB of each multi-energy generator of the plurality of multi-energy generators; and
thermally insulated thermal conductors that extend through the lumens and are thermally coupled to the frame and the heat sink of each multi-energy generator of the plurality of multi-energy generators.

30. The system of claim 29, wherein:
the plurality of different energy types comprise a vibratory energy; and
the plurality of generator elements of each multi-energy generator of the plurality of multi-energy generators comprise an electromechanical actuator that is operable to output the vibratory energy and comprises a skin-facing side that is positioned on or adjacent the skin of the muscoskeletal joint when the brace is worn.

31. The system of claim 21, wherein:
the plurality of different energy types comprise one or more of an electrical energy, a light energy, a thermal energy, a pressure energy, and a vibratory energy; and
each generator element of the plurality of generator elements of each multi-energy generator of the plurality of multi-energy generators comprises circuitry that is independently operable to output the one or more of the electrical energy, the light energy, the thermal energy, the pressure energy, and the vibratory energy.

32. The system of claim 21, comprising:
a power source that is mounted to the frames and operable with the plurality of generator elements to output the plurality of different energy types;
a controller operable with the power source to cause the plurality of multi-energy generators to output the plurality of different energy types;
one or more usage sensors operable with the power source and the controller to output usage data associated with the brace; and
one or more efficacy sensors operable with the power source and the controller to output physiological data associated with the muscoskeletal joint.

33. A system comprising:
a brace that is wearable on a limb, the brace comprising frames and hinges operable to guide and support movements of a muscoskeletal joint of the limb by restricting rotational movements of the limb when the brace is worn;
a plurality of multi-energy generators that are attachable to the brace and positionable at different treatment sites about the muscoskeletal joint when the brace is worn, each multi-energy generator of the plurality of multi-energy generators comprising:
a plurality of generator elements operable to output a plurality of different energy types in a signal direction toward the muscoskeletal joint at one treatment site of the different treatment sites, each generator element of the plurality of generator elements being independently operable to output one energy type of the plurality of different energy types, and
a heat sink that dissipates excess heat generated from the plurality of generator elements into air surrounding the limb; and
a lattice structure comprising a plurality of cords, a plurality of nodes, and a plurality of openings,
the plurality of cords forming an elastic open framework that structurally interconnects the plurality of nodes to one another and the frame,
the plurality of nodes containing the plurality of multi-energy generators and being operable with the plurality of cords to attach the plurality of multi-energy generators to the frames and, when the brace is worn, to press skin-facing sides of the plurality of multi-energy generators toward the different treatment sites during the movements of the muscoskeletal joint,
the plurality of openings being located between the plurality of nodes and sized to permit injection of a treatment fluid into the muscoskeletal joint at locations below the different treatment sites when the brace is worn,
an exterior surface of each node of the plurality of nodes being operable with a needle to inject the treatment fluid into the locations through one or more openings of the plurality of openings without removing the brace.

34. The system of claim 33, wherein:
the frames comprise a first beam with a first 3D curved shape that wraps partially around a first portion of the limb on a first side of the muscoskeletal joint and a second beam with a second 3D curved shape that wraps partially around a second portion of the limb on a second side of the muscoskeletal joint; and
the hinges are operable with the first beam and the second beam to guide and support the movements of the muscoskeletal joint by limiting rotational movements of the first portion of the limb relative to the second portion of the limb during the movements.

35. The system of claim 33, wherein:
the limb is a leg;
the muscoskeletal joint is a knee of the leg comprising a patella, an ACL, an MCL, and a pes anserine; and
the different treatment sites are located approximate to the patella, the ACL, the MCL, and the pes anserine.

36. The system of claim 35, comprising a sleeve that is:
separate from the brace;
wearable under the brace; and
comprises sleeve openings that are located approximate to the different treatment sites and sized to position skin-facing sides of the plurality of multi-energy generators against skin of the muscoskeletal joint when the sleeve is worn under the brace.

37. The system of claim 36, wherein:
the sleeve openings comprise:
a plurality of interface openings sized to receive the skin-contacting surfaces of the plurality of multi-generators; and
at least one injection opening that is located to permit injection of a fluid through the sleeve and into the patella, the ACL, the MCL, or the pes anserine when the brace is worn.

38. The system of claim 33, wherein:
each heat sink comprises a backing plate and a plurality of fins extending outwardly from the backing plate; and
each multi-energy generator of the plurality of multi-energy generators is thermally coupled to the backing plate.

39. The system of claim 38, wherein:
the plurality of different energy types comprise a thermal energy comprising hot or cold; and
the plurality of generator elements of each multi-energy generator of the plurality of multi-energy generators comprise a thermoelectric device with:
a skin-facing side that is positioned on skin of the muscoskeletal joint and operable to output one of the hot or cold to the skin when the brace is worn; and
an outward-facing side that is thermally coupled to the backing plate and operable to dissipate the other one of the hot or cold into the air surrounding the limb via a surface area of the plurality of fins.

40. The system of claim 38, wherein:
the plurality of different energy types comprise a vibratory energy; and
the plurality of generator elements of each multi-energy generator of the plurality of multi-energy generators comprise an electromechanical actuator that is operable to output the vibratory energy and comprises a skin-facing side that is positioned on or adjacent the skin of the muscoskeletal joint when the brace is worn.

41. The system of claim 33, wherein:
the plurality of different energy types comprise one or more of an electrical energy, a light energy, a thermal energy, a pressure energy, and a vibratory energy; and
each generator element of the plurality of generator elements of each multi-energy generator of the plurality of multi-energy generators comprises circuitry that is independently operable to output the one or more of the electrical energy, the light energy, the thermal energy, the pressure energy, and the vibratory energy.

42. The system of claim 33, comprising:
a treatment delivery device operable with the needle and the exterior surfaces of the plurality of nodes to deliver the treatment fluid to the locations through one or more openings of the plurality of openings,
wherein the treatment delivery device comprises a body defining:
a contact surface that is positionable against the exterior surfaces of the plurality of nodes and slidable around a central axis of the exterior surfaces; and
a needle guide operable to guide a tip of the needle into the locations along an injection axis that intersects the central axis when the contact surface is positioned against the exterior surfaces.

43. The system of claim 42, wherein the contact surface is curved to match a curvature of the exterior surfaces.

44. The system of claim 43, wherein the treatment fluid comprises one of a biologic treatment, a chemical treatment, and a pharmacological treatment.

45. The system of claim 42, wherein the treatment fluid is affected by at least one energy type of the plurality of different energy types.

46. The system of claim 45, wherein:
the treatment fluid comprises:
a biocompatible gel, and
a plurality of microspheres containing separate portions of the treatment fluid; and
each microsphere of the plurality of microspheres is operable to release its separate portion of the treatment fluid responsive to at least one energy type of the plurality of different energy types.

47. The system of claim 46, wherein the plurality of microspheres comprise:
a first set of microspheres operable to release a first volume of the treatment fluid responsive to an output of a first energy type of the plurality of different energy types; and
a second set of microspheres operable to release a second volume of the treatment fluid responsive to either:
a different output of the first energy type, or
an output of a second energy type of the plurality of different energy types.

48. The system of claim 46, comprising:
a power source that is mounted to the frames and operable with the plurality of generator elements to output the plurality of different energy types;
a controller operable with the power source to cause to the plurality of microspheres to release their separate portions of the treatment fluid by causing the plurality of multi-energy generators to output the plurality of different energy types;
one or more usage sensors operable with the power source and the controller to output usage data associated with the brace; and
one or more efficacy sensors operable with the power source and the controller to output physiological data associated with the muscoskeletal joint.
